(19) United States
(12) Patent Application Publication
(10) Pub. No.: US 2025/0010335 A1
(43) Pub. Date: Jan. 9, 2025

(54) METHODS AND SYSTEMS FOR AN ELECTRO-ACOUSTIC TRANSDUCER

(71) Applicant: GE Precision Healthcare LLC, Waukesha, WI (US)

(72) Inventors: Jean-Luc Diot, Valbonne (FR); Omid Farhanieh, Valbonne (FR); Edouard Da Cruz, Valbonne (FR)

(21) Appl. No.: 18/349,054

(22) Filed: Jul. 7, 2023

(51) Int. Cl. B06B 1/02; A61B 8/00

(52) U.S. Cl. CPC B06B 1/0292; A61B 8/4483; B06B 2201/76

(57) ABSTRACT

Various methods and systems are provided for an electroacoustic module (EAM) for a probe. In one example, the EAM may be formed of a microelectromechanical systems (MEMS) wafer bonded to a complementary metal-oxide semiconductor (CMOS) wafer and having a front side formed of a surface of the MEMS wafer. The surface of the MEMS wafer may include an active area of capacitive micromachined ultrasound transducer (CMUT) cells and input/output (I/O) regions of output contacts arranged adjacent to the active area.

### FIELD

Embodiments of the subject matter disclosed herein relate to a transducer for medical imaging.

### BACKGROUND

A microelectromechanical systems (MEMS) ultrasound device (hereafter, MEMS device) may be used for imaging and/or therapy targets such as organs and soft tissues in a human body, as well as non-human targets. For example, the MEMS device may be used for applications such as ultrasound/acoustic sensing, non-destructive evaluation (NDE), ultrasound therapy (e.g., High Intensity Focused Ultrasound (HIFU)), etc., in addition to ultrasound imaging of humans, animals, etc. The MEMS device may include both electronic and moving parts and may be formed from components within a size range of 0.001 to 0.3 mm.

MEMS devices may use real time, non-invasive high frequency (e.g., in a range of 100 KHz to tens of MHz) sound waves to produce a series of two-dimensional (2D) and/or three-dimensional (3D) images. The sound waves may be transmitted by a transmit transducer, and the reflections of the transmitted sound waves may be received by a receive transducer. The received sound waves may then be processed to display an image of the target. For some types of MEMS devices used as a transmit transducer and/or a receive transducer, such as a capacitive micromachined ultrasound transducer (CMUT), the CMUT may include a top electrode and a bottom electrode, which may be separated by a gap or a cavity.

In a probe used for medical imaging, such as an ultrasound probe, a CMUT array may be coupled to electronic circuits, such as application-specific integrated circuits (ASICs). The ASICs may have a configuration that is dedicated to a specific CMUT array and fabricating ASICs with new configurations to accommodate different CMUT arrays may be costly and laborious. It may therefore be desirable to enable adapt already existing ASIC die types for use with different CMUT array arrangements.

### BRIEF DESCRIPTION

In one embodiment, an ultrasound probe comprises an electroacoustic module formed of a microelectromechanical systems (MEMS) wafer bonded to a complementary metal-oxide semiconductor (CMOS) wafer as an Application-Specific Integrated Circuit (ASIC) and having a front side formed of a surface of the MEMS wafer, the surface of the MEMS wafer including an active area of capacitive micromachined ultrasound transducer (CMUT) cells and input/output (I/O) regions of input and output contacts of ASIC which are redistributed to the surface of the MEMS wafer arranged adjacent to the active area. In this way, the electroacoustic module may be provided using a wafer-scale manufacturing approach that allows different CMUT wafer configurations to be adapted to a given ASIC structure.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a block diagram of an exemplary ultrasound system that may be used in ultrasound imaging, according to an embodiment;

FIG. 2 shows a cross-sectional view of a CMUT cell, according to an embodiment;

FIG. 3 shows an example of an ASIC wafer that may be diced and used to form tiles of an electroacoustic module (EAM);

FIG. 4 shows a conventional configuration of ASIC tiles formed from the ASIC wafer of FIG. 3;

FIG. 5 shows a front side of an extended EAM tile, according to an embodiment;

FIG. 6 shows a final configuration of the extended EAM tile of FIG. 5, according to an embodiment;

FIG. 7 shows an EAM formed of the extended EAM tile of FIG. 6, according to an embodiment;

FIG. 8 shows a first cross-sectional view of the extended EAM tile of FIG. 5, according to an embodiment;

FIG. 9 shows a second cross-sectional view of the extended EAM tile of FIG. 5, according to an embodiment;

FIG. 10 shows a method for fabricating an extended EAM tile using a wafer-scale, semiconductor approach, according to an embodiment;

FIG. 11 shows coupling of a silicon-on-insulator (SOI) to a highly doped silicon wafer, which may be included in the method of FIG. 10, according to an embodiment;

FIG. 12 shows a CMUT wafer formed by the coupling of the SOI to the highly doped silicon wafer of FIG. 11, according to an embodiment;

FIG. 13 shows coupling of the CMUT wafer of FIG. 12 to an ASIC die, according to an embodiment; and

FIG. 14 shows a configuration of the extended EAM tile formed via the method of FIG. 10, according to an embodiment.

### DETAILED DESCRIPTION

The following description relates to various embodiments of a transducer probe (e.g., an ultrasound probe), and more specifically a process for manufacturing a CMUT array using a complementary metal-oxide semiconductor (CMOS) approach. The process described herein may allow input/output (I/O) connections of an electro-acoustic module (EAM) to be redistributed to achieve a desired positioning and alignment of the EAM components. The EAM may include CMUT arrays coupled to ASIC tiles or dies, where the EAM may be fabricated using CMOS technology to achieve wafer-to-wafer bonding between a CMUT wafer and a CMOS wafer. The CMOS wafer may be diced and tiled to form the ASIC dies having extended footprints (e.g., enlarged areas).

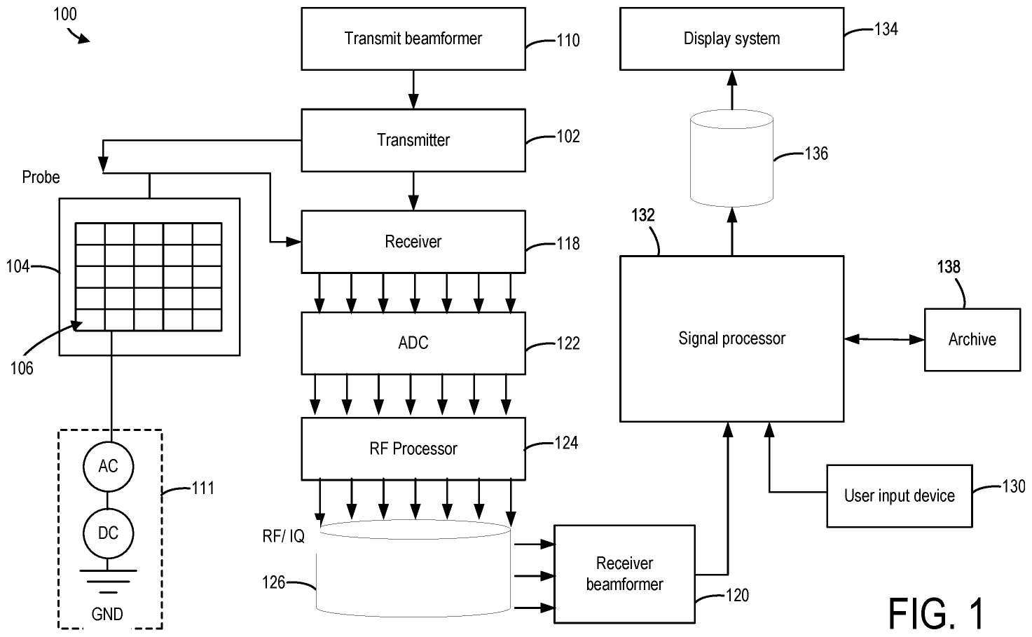

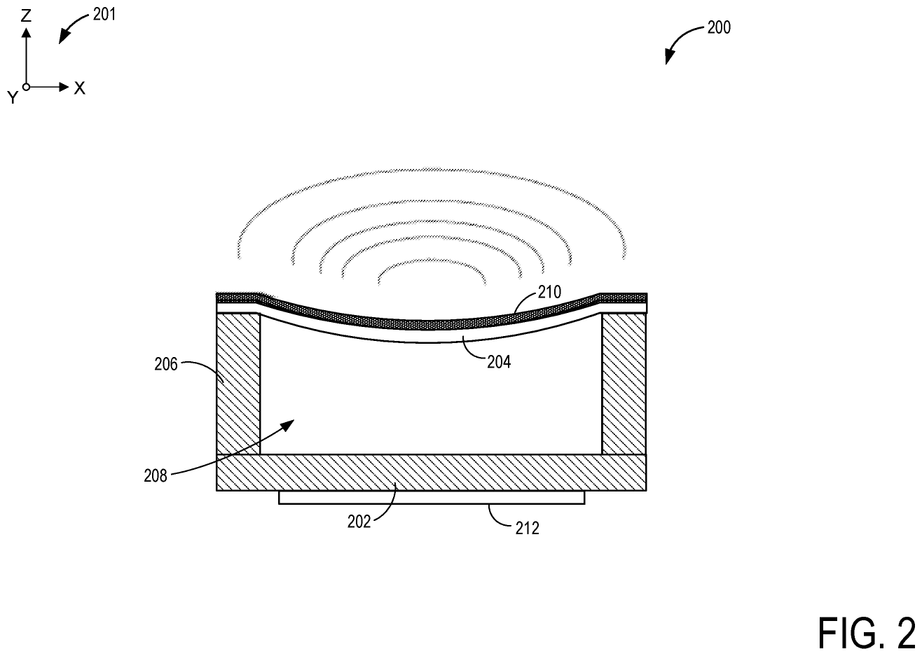

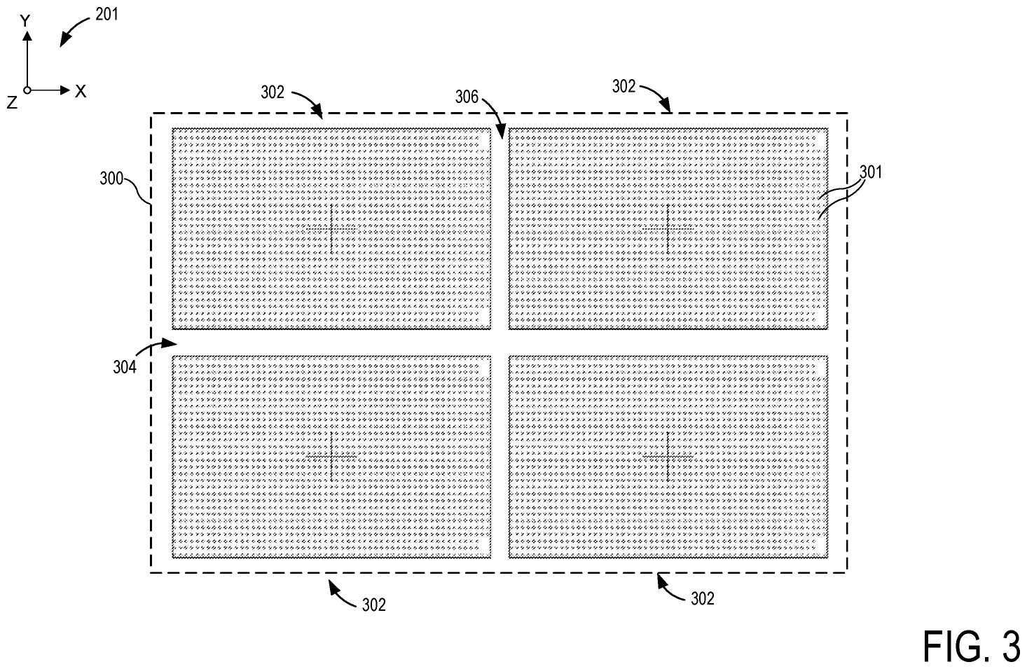

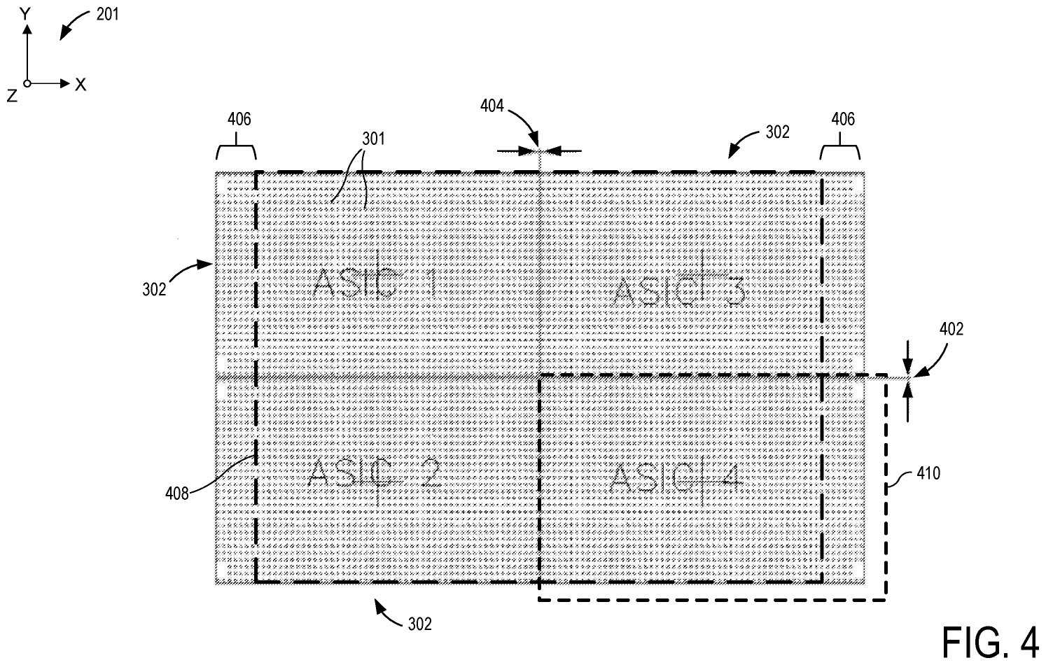

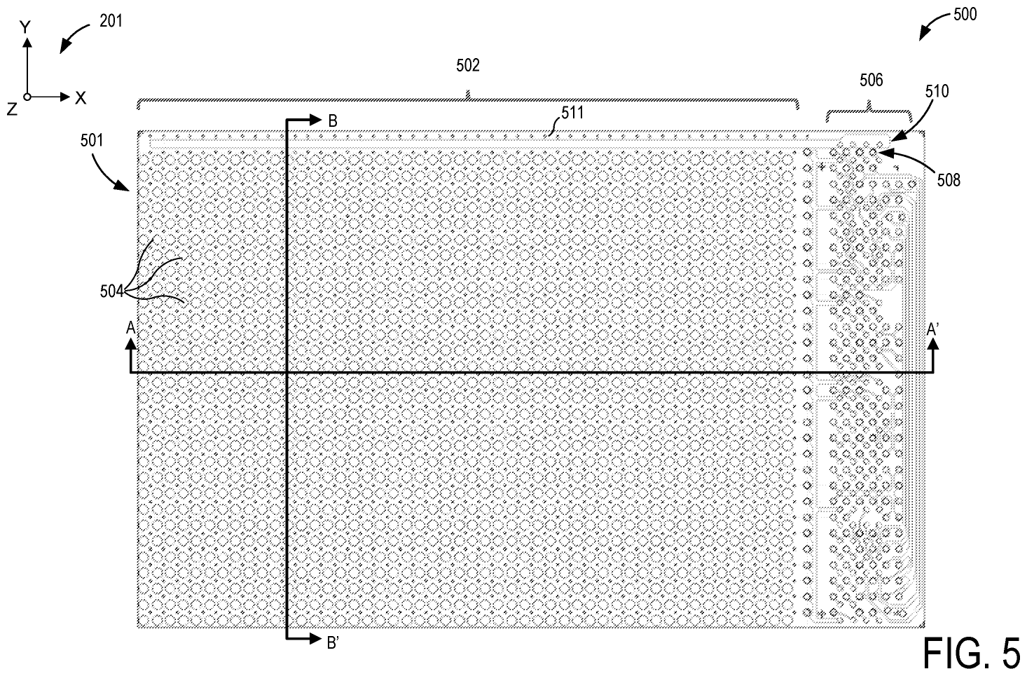

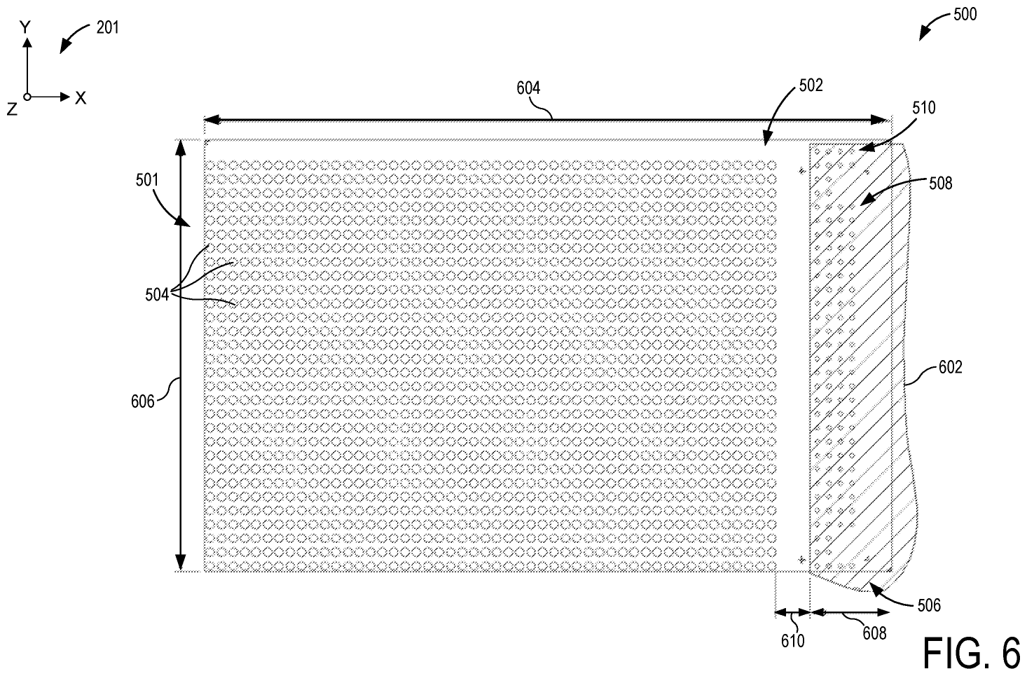

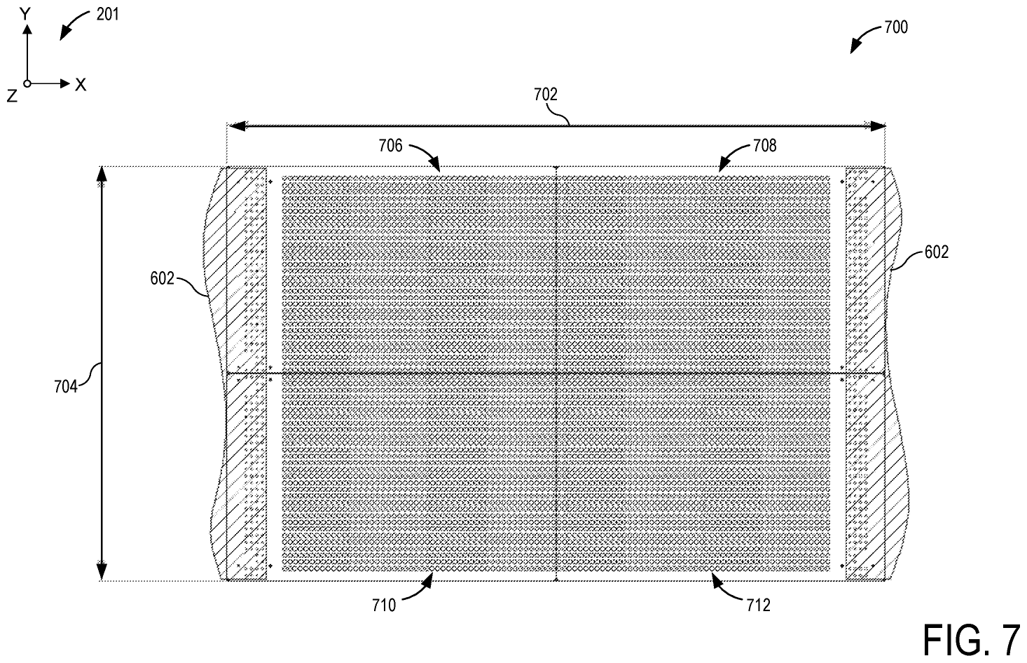

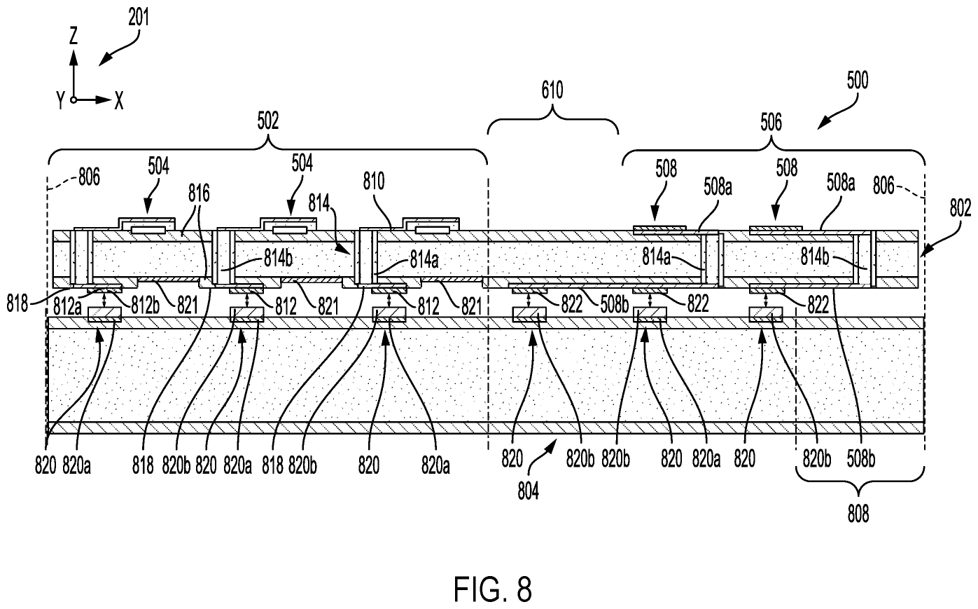

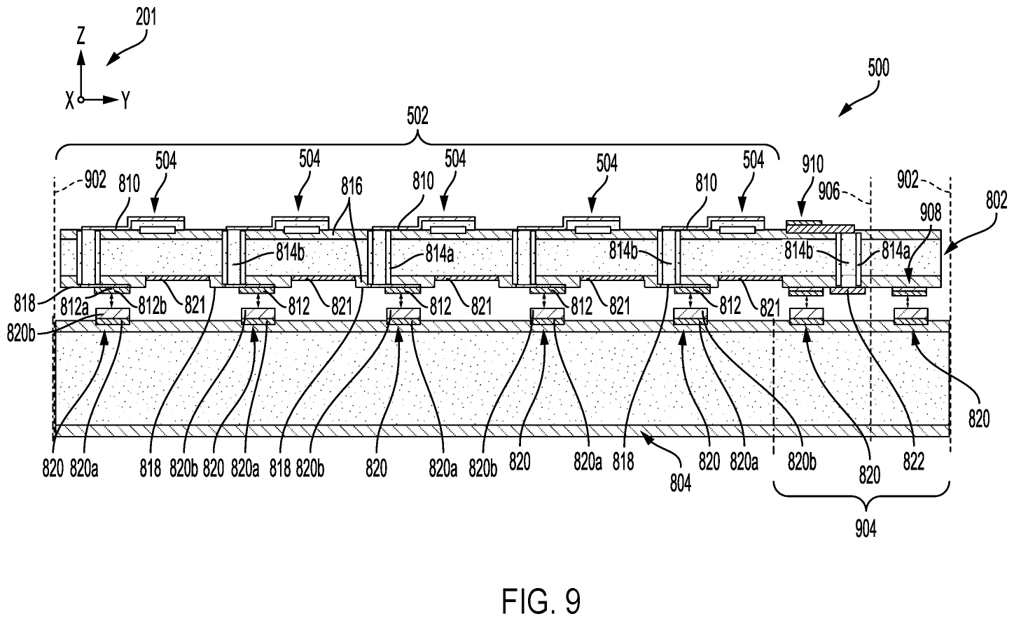

The EAM may be a device for an ultrasound imaging system, as one example, such as the exemplary ultrasound imaging system depicted in FIG. 1. The EAM may include a plurality of CMUT cells, as shown in FIG. 2, which may be incorporated in a CMUT array. Each CMUT array may be coupled to at least one ASIC tile which may be diced from a wafer, where each paired CMUT array and ASIC die forms an EAM tile. In conventional systems, as shown in FIG. 3, the ASIC wafer may be diced with dicing lanes to form ASIC tiles which are coupled to a CMUT die to form the EAM tile. In one example, as described herein, the dicing lanes may be leveraged to redistribute I/O connections by extending the ASIC die areas to include the dicing lanes, as indicated in FIG. 4. The ASIC dies may be coupled to the CMUT arrays to form an EAM with I/O connection accessibility at a front side of the EAM. An EAM tile of the EAM is illustrated with and without an interconnecting circuit, such as a flex circuit, in FIGS. 5 and 6, respectively. A final configuration of the EAM is shown in FIG. 7. Cross-sectional views of the EAM tile of FIGS. 5 and 6 are depicted in FIGS. 8 and 9, illustrating direct coupling of the CMUT device to the ASIC without interfacing layers, such as an interposer and/or flex circuits, arranged between the CMUT and the ASIC.

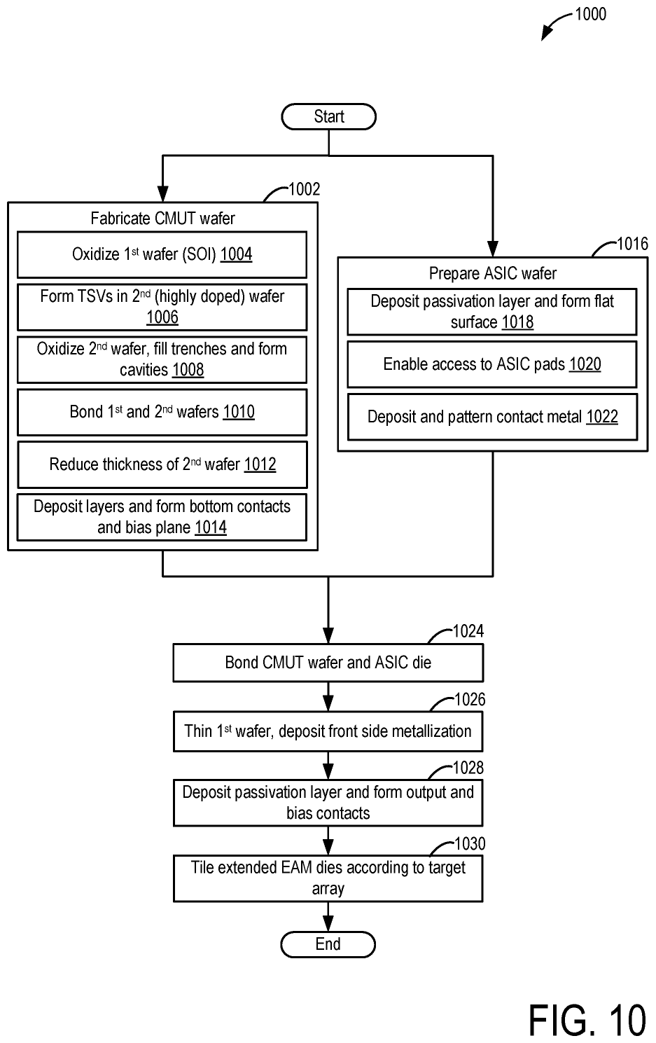

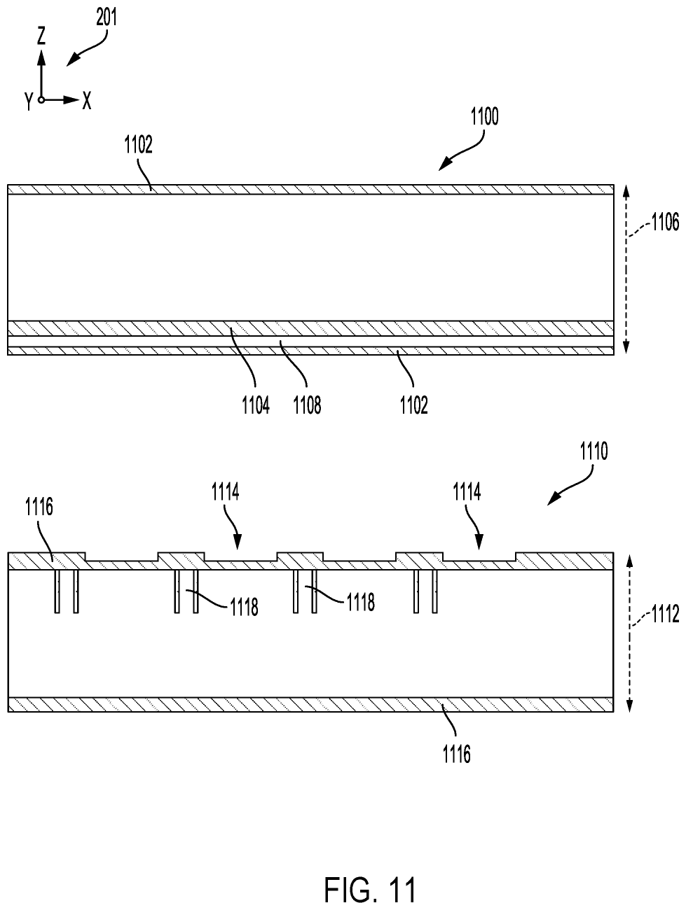

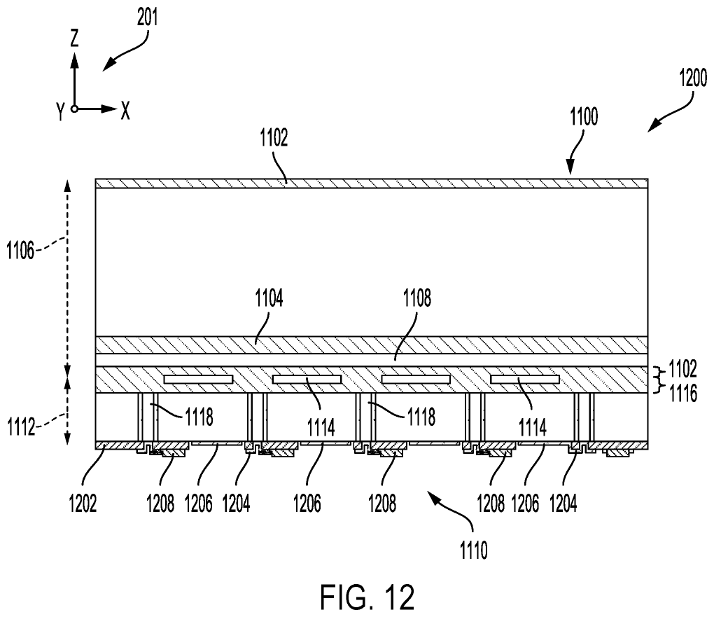

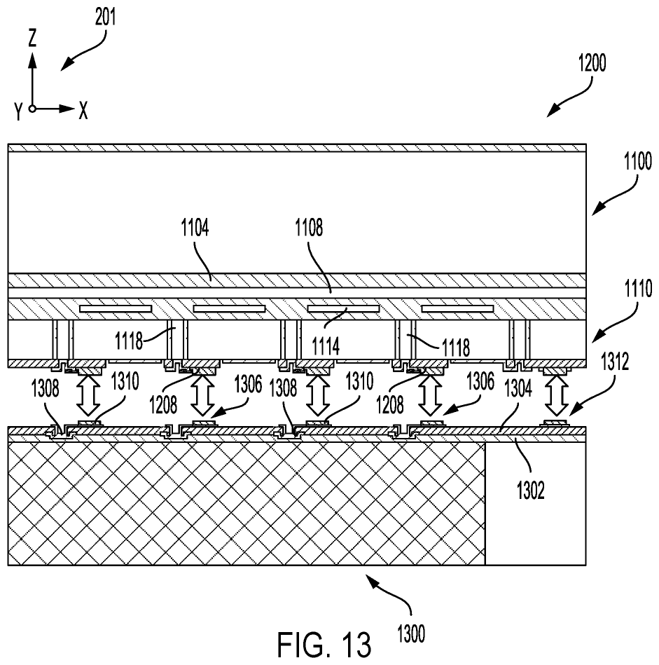

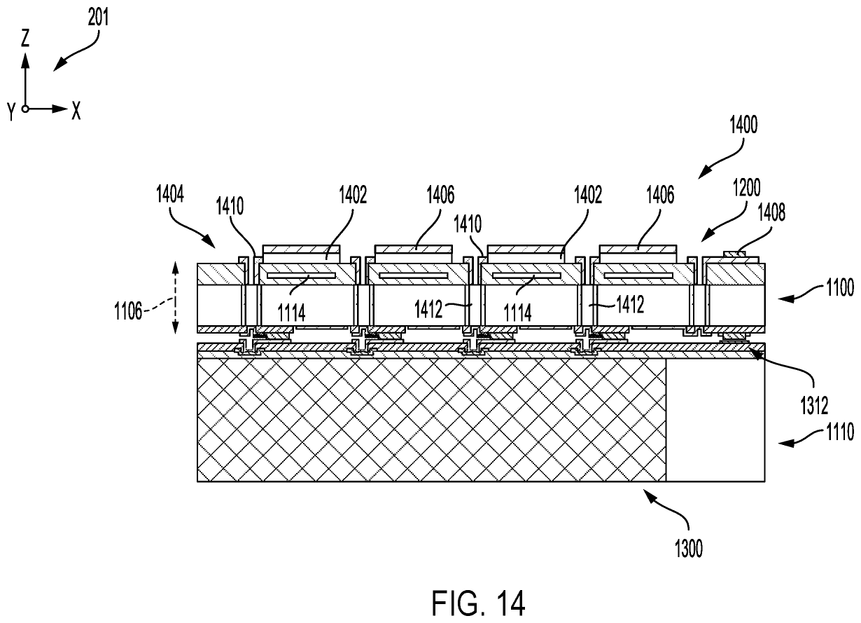

A method for fabricating the EAM via a wafer-scale, semiconductor approach is shown in FIG. 10 and steps included in the method are illustrated in FIGS. 11-14. For example, the wafer-scale approach may include semiconductor processing techniques such as bonding of a silicon-on-insulator (SOI) wafer to a highly doped silicon wafer, as shown in FIG. 11, to form a CMUT wafer depicted in FIG. 12. A CMOS wafer may be coupled to the CMUT wafer, as illustrated in FIG. 13, and processed to form an extended EAM tile shown in FIG. 14.

As used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes ofB-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

FIG. 1 is a block diagram of an exemplary ultrasound system that may be used in ultrasound imaging, in accordance with various embodiments. Referring to FIG. 1, there is shown a block diagram of an exemplary ultrasound system 100. The ultrasound system 100 comprises a transmitter 102, an ultrasound probe 104, a transmit beamformer 110, a receiver 118, a receive beamformer 120, A/D converters 122, an RF processor 124, an RF/IQ buffer 126, a user input device 130, a signal processor 132, an image buffer 136, a display system 134, and an archive 138. The circuit 111 is a non-limiting example of bias of a CMUT and variations in a configuration of the circuit 111 are possible without departing from the scope of the present disclosure.

The transmitter 102 may comprise suitable logic, circuitry, interfaces and/or code that may be operable to drive the ultrasound probe 104. The ultrasound probe 104 may comprise, for example, a single element CMUT, a 1D array of CMUTs, 2D array of CMUTs, an annular (ring) array of CMUTs, etc. Accordingly, the ultrasound probe 104 may comprise a group of transducer elements 106 that may be, for example, CMUTs. In certain embodiments, the ultrasound probe 104 may be operable to acquire ultrasound image data covering, for example, at least a substantial portion of an anatomy, such as the heart, a blood vessel, or any suitable anatomical structure. Each of the transducer elements 106 may be referred to as a channel.

The transmit beamformer 110 may comprise suitable logic, circuitry, interfaces and/or code that may be operable to control the transmitter 102 that drives the group of transducer elements 106 to emit ultrasonic transmit signals into a region of interest (e.g., human, animal, underground cavity, physical structure and the like). The transmitted ultrasonic signals may be backscattered from structures in the object of interest, like blood cells or tissue, to produce echoes. The echoes can then be received by the transducer elements 106. For example, one or more drive circuits 111 may be coupled to and drive or control the electrodes of each transducer element 106. For example, the one or more drive circuits may be coupled to separate AC and DC voltage sources.

The group of transducer elements 106 in the ultrasound probe 104 may be operable to convert the received echoes into analog signals and communicated to a receiver 118. The receiver 118 may comprise suitable logic, circuitry, interfaces and/or code that may be operable to receive the signals from the ultrasound probe 104. The analog signals may be communicated to one or more of the plurality of A/D converters 122.

Accordingly, the ultrasound system 100 may multiplex such that ultrasonic transmit signals are transmitted during certain time periods and echoes of those ultrasonic signals are received during other time periods. Although not shown explicitly, various embodiments of the disclosure may allow concurrent transmission of ultrasonic signals and reception of echoes from those signals. In such cases, the probe may comprise transmit transducer elements and receive transducer elements.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF data, which may be, for example, I/Q signal data, real valued RF data, etc., may then be communicated to an RF/IQ buffer 126. The RF/IQ buffer 126 may comprise suitable logic, circuitry, interfaces and/or code that may be operable to provide temporary storage of the RF or 1/Q signal data, which is generated by the RF processor 124.

Accordingly, various embodiments may have, for example, the RF processor 124 process real valued RF data, or any other equivalent representation of the data, with an appropriate RF buffer 126. The receive beamformer 120 may comprise suitable logic, circuitry, interfaces and/or code that may be operable to perform digital beamforming processing to sum, for example, delayed, phase shifted, and/or weighted channel signals received from the RF processor 124 via the RF/IQ buffer 126 and output a beam summed signal. The delayed, phase shifted, and/or weighted channel data may be summed to form a scan line output from the receive beamformer 120, where the scan line may be, for example, complex valued or non-complex valued. The specific delay for a channel may be provided, for example, by the RF processor 124 or any other processor configured to perform the task. The delayed, phase shifted, and/or weighted channel data may be referred to as delay aligned channel data.

The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 may comprise a plurality of receive beamformers 120.

The user input device 130 may be utilized to input patient data, scan parameters, settings, select protocols and/or templates, and the like. In an exemplary embodiment, the user input device 130 may be operable to configure, manage, and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include switch(es), button(s), rotary encoder(s), a touchscreen, motion tracking, voice recognition, a mouse device, keyboard, camera, and/or any other device capable of receiving a user directive. In certain embodiments, one or more of the user input devices 130 may be integrated into other components, such as the display system 134 or the ultrasound probe 104, for example. As an example, user input device 130 may comprise a touchscreen display.

The signal processor 132 may comprise suitable logic, circuitry, interfaces and/or code that may be operable to process ultrasound scan data (e.g., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in a live or off-line operation. In various embodiments, the processed image data can be presented at the display system 134 and/or stored at the archive 138. The archive 138 may be a local archive, a Picture Archiving and Communication System (PACS), or any suitable device for storing images and related information.

The signal processor 132 may comprise one or more central processing units, microprocessors, microcontrollers, and/or the like. The signal processor 132 may be an integrated component, or may be distributed across various locations, for example. In an exemplary embodiment, the signal processor 132 may be capable of receiving input information from the user input device 130 and/or the archive 138, generating an output displayable by the display system 134, and manipulating the output in response to input information from the user input device 130, among other things. The signal processor 132 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates may range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes worth of frames of ultrasound scan data. The frames of ultrasound scan data are stored in a manner to facilitate retrieval thereof according to its order or time of acquisition. The image buffer 136 may be embodied as any known data storage medium.

The display system 134 may be any device capable of communicating visual information to a user. For example, a display system 134 may include a liquid crystal display, a light emitting diode display, and/or any suitable display or displays. The display system 134 can be operable to present ultrasound images and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display. Additionally, while the ultrasound system 100 was described to comprise one receive beamformer 120, one RF processor 124, and one signal processor 132, various embodiments of the disclosure may use various number of processors. For example, various devices that execute code may be referred to generally as processors. Various embodiments may refer to each of these devices, including each of the RF processor 124 and the signal processor 132, as a processor. Furthermore, there may be other processors to additionally perform the tasks described as being performed by these devices, including the receive beamformer 120, the RF processor 124, and the signal processor 132, and all of these processors may be referred to as a “processor” for ease of description.

As described above, the ultrasound probe may include an array of CMUTs which transmit or receive the ultrasonic waves based on their electrostatic principle. An example of a CMUT cell 200 is depicted in FIG. 2. A set of references axes 201 is provided, including an x-axis, a y-axis, and a z-axis. In one example, the z-axis may be parallel with a direction of acoustic signal transmission. It will be appreciated that the CMUT cell 200 is a simplified, representative embodiment, and other examples may vary in configuration.

The CMUT cell 200 may be one of an array of CMUT cells which may be fabricated on a substrate 202, such as a heavily doped silicon wafer. For each CMUT cell 200, a thin membrane or diaphragm 204 is suspended above the substrate 202. The membrane 204 may be formed of silicon oxide, for example, and may be supported on its periphery by an insulating support 206, which may be composed of silicon oxide or silicon nitride. A cavity 208 between the membrane 204 and the substrate 202 may be air- or gas-filled or entirely or partially evacuated. A film or layer of conductive material, such as aluminum alloy or another suitable conductive material, may form a first electrode 210 on the membrane 204. Another film or layer of conductive material may form a second electrode 212 at the substrate 202. For example, the substrate 202 may be oxidized and the second electrode 212 may be deposited along an outer surface of the substrate 202 and along a bottom, with respect to the z-axis, of the CMUT cell 200. Alternatively, the second electrode 212 may be formed by suitable doping of the semiconducting substrate 202. By positioning the second electrode 212 at the bottom of the CMUT cell 200, outside of the cavity 208, the second electrode 212 may be connected to an external circuit.

The first and second electrodes 210, 212, which are separated at least by the cavity 208, may form a capacitance. When an impinging acoustic signal causes the membrane 204 to vibrate, a variation in the capacitance may be detected using electronics coupled to the CMUT cell 200. The acoustic signal may thereby be transduced into an electrical signal which may be transmitted to other electronic components of an imaging system, as shown in FIG. 1, via an electrical circuit coupled to the CMUT cell 200. For example, the electrical circuit may be at least one ASIC die, which may also be arranged in an array in a transducer probe, such as the probe 104 of FIG. 1.

A CMUT array and an ASIC die may be included in an EAM of an ultrasound probe. The ASIC die may have a dedicated configuration, e.g., dimensions and positioning of I/O connections and supply pads, to couple to a CMUT array for a given probe type. Use of the ASIC die is thereby constrained to the given probe type and corresponding CMUT array. Manufacturing of a different ASIC die is therefore required for use in any new (e.g., different) probe and CMUT array configuration, which may demand prolonged development times and high costs.

To broaden a use of a given ASIC die, e.g., to allow the ASIC die to be used with more than one type of probe and CMUT array, the EAM may be fabricated via a process that incorporates CMOS technology. For example, ASIC dies may be formed form a CMOS wafer which are then bonded to CMUT wafers to create the EAM. This approach may allow dicing lanes of the CMOS wafer to removed or precluded, thereby extending dimensions of the ASIC dies (hereafter also referred to as extended ASIC dies). The extended ASIC dies may enable rearrangement of I/O connections and bias connections to a front side of the EAM, through the CMUT wafers. As a result, an EAM may be manufactured via a low cost, scalable approach that enables simplified and more accessible positioning of the I/O and bias connections. The systems and methods described herein also allow a given ASIC configuration to be used with various CMUT arrays, obviating a demand for fabrication of new ASIC dies for different probe types. A given ASIC die may thereby be compatible for electrical coupling with more than one configuration of a CMUT array.

To illustrate differences between conventional processing of ASIC tiles for EAMs and formation of extended ASIC dies via wafer-scale processing, a conventional configuration of an ASIC wafer 300 is depicted in an initial stage before dicing in FIG. 3 and in a final configuration in FIG. 4. A front side of the ASIC wafer 300 is viewed in FIGS. 3, illustrating a plurality of contacts 301 distributed across the front side. Each of the plurality of contacts 301 may pilot a CMUT cell of a monolithic CMUT die coupled to and stacked over ASIC die or tiles 302 formed from the ASIC wafer 300. In one example, the ASIC wafer 300 may, after processing to form the ASIC dies 302, be coupled to the CMUT die, which includes a transducer array, through more than one interfacing layer. For example, the transducer array may be coupled to an interposer flex (e.g., flex circuit) and the ASIC tiles 302 may be coupled to an ASIC flex. The flexes may be coupled to one another to provide electric connectivity between the transducer array at the front side of the EAM and the ASIC die at a back side of the ASIC tiles 302. A complexity of the flexes may add to an overall cost of an EAM.

During fabrication, the ASIC wafer 300 may be double diced with a first dicing lane 304 and a second dicing lane 306 to form the individual ASIC dies 302. The first dicing lane 304 may be parallel with the x-axis and the second dicing lane 306 may be parallel with the y-axis. The dicing lanes may remove unused regions of the ASIC wafer 303, separating and electrically isolating the ASIC dies 302 from one another.

After double dicing of the ASIC wafer 303 and removal of the first and second dicing lanes 304, 306, the ASIC dies 302 may be rearranged as shown in FIG. 4. The ASIC dies 302 may be positioned proximate to one another with a target minimum spacing in-between. For example, a first distance 402 between the ASIC dies 302 along the y-axis may be 0.038 mm and a second distance 404 between the ASIC die 302 along the x-axis may be 0.013 mm.

The relative positioning of the ASIC dies 302 and stacking of the MEMS device (CMUT die) over the ASIC dies 302 may render I/O connections of the ASIC dies 302 to be inaccessible from the front side of the EAM. Output I/O connections may be located at outer perimeters 406 of the ASIC tiles 302 (and of the EAM) and may be routed outwards from the ASIC tiles 302, as a result. For example, for the I/O connections of the ASIC transferred to the top of the EAM via RDL, electrical interconnections may be formed via wire bonding which may extend outwards from an outer perimeter 406 of the EAM. The outer perimeters 406 of the EAM may be formed of regions of the ASIC tiles 302 that extend beyond a perimeter 408 of the monolithic CMUT die, which may be stacked over the ASIC tiles arrangement of FIG. 4.

In one example, dicing of the ASIC wafer (e.g., the ASIC wafer 300 of FIG. 3) may be modified to redistribute the I/O connections and bias connections and enable the I/O connections to be more readily accessible. For example, dimensions of the ASIC tiles 302, e.g., along the y-x plane, may be extended at least along one direction by retaining at least one dicing lane between the ASIC tiles 302. This may be achieved by singly dicing the ASIC wafer along each of the y- and x-axes, instead of double dicing. The ASIC tiles 302 may be formed and separated without removing at least one of the first and second dicing lanes 304, 306 of FIG. 3. As one example, the first dicing lane 304 may be retained while the second dicing lanes 306 may be removed. In other examples, both the first and the second dicing lanes 304, 306 may be retained, at least partially. As such, a usable area of the ASIC tiles 302 may be extended along at least the y-axis and, in some examples, both the y- and x-axes, without altering the arrangement of the plurality of contacts 301. In one example, extension of the ASIC tiles may be leveraged to incorporate CMUT bias contacts thereat, which may otherwise be positioned around an outermost perimeter of the CMUT die.

For example, the ASIC tiles 302 may be extended to have a footprint 410, as indicated in FIG. 4. A CMUT wafer may also be diced into die having dimensions corresponding to the footprint 410 of the ASIC tiles 302. This may be achieved by fabricating an EAM via a semiconductor approach, in which the CMUT wafer is fabricated according to a configuration of a CMOS wafer as long as the acoustic requirements fit, the CMOS wafer being an ASIC wafer. The CMUT wafer and the CMOS wafer may be coupled to one another and then further processed to form tiles of the EAM. Details of the EAM tiles and the semiconductor approached to manufacturing of the EAM tiles are provided herein with reference to FIGS. 5-14.

As an example of an EAM having extended dies as described above, an extended EAM die 500 of the EAM is shown in FIG. 5 from a top-down view, showing details of a front side 501 of the extended EAM die 500, formed of a front-facing surface of a CMUT die. The extended EAM tile 500 may be formed by singly dicing an EAM stack formed of a CMOS wafer coupled to a CMUT wafer to separate the EAM stack into EAM dies, each EAM die formed of a CMUT die stacked over an ASIC die. The extended EAM die 500 is depicted prior to incorporation of an interconnecting circuit, such as a flex circuit, which may be added after fabrication of the extended EAM die 500 to form the EAM.

The extended EAM die 500 includes an active area 502 formed of an array of transducers, where the transducers may be CMUT cells 504 (hereafter, CMUTs). Each of the CMUTs 504 may be similarly configured to the CMUT cell 200 of FIG. 2, for example. The extended EAM die 500 may also include an I/O region 506, which includes output contacts 508 and bias contacts 510. As such, the bias contacts are redistributed to the I/O region 506, rather than arranged around an outer perimeter of an active area of a CMUT die having a conventional arrangement. The bias contacts 510 may be electrically coupled to a bias metal plate 511 that extends across the extended EAM die 500 along the x-axis, proximate to an upper edge (with respect to the y-axis) of the extended EAM die 500. Optional bias vias may extend through the CMUT die hosting the CMUTs 504, under the bias metal plate 511, with respect to the z-axis, to couple the bias metal plate 511 to bias planes located at an underside of the CMUT wafer. The output contacts 508 may enable electrical signals to be transmitted from the extended EAM die 500 to other electrical components of an imaging system while the bias contacts 510 may allow a bias voltage to be applied to the CMUT cells 504. The I/O region 506 may form an area adjacent, e.g., next to or beside, the active area 502 along the x-axis, at the front side of the CMUT die. Further, the I/O region 506 may be located along a periphery or outer edge of the EAM relative to the x-axis when multiple EAM dies are arranged to form a tile.

The active area 502 may occupy a larger area of the extended EAM die 500 than the I/O region. In one example, the active area 502 may include 50 columns (e.g., as aligned along the y-axis) of the CMUTs 504 while the I/O region 506 may include at least 4 columns (as shown more clearly in FIG. 6) of the output contacts 508. By extending the dimensions of the extended EAM die 500, relative to the ASIC die 302 of FIGS. 3 and 4, the output contacts 508 may be redistributed to the front side 501 of the extended EAM die 500 (e.g., at a front-facing surface of the CMUT die), in the I/O region 506 instead of being positioned at the outer perimeters (e.g., the outer perimeters 406 of FIG. 4) of the ASIC tiles that extend beyond a perimeter of the monolithic CMUT die in the conventional EAM arrangement. Further, the CMUT wafer may be directly coupled to the ASIC wafer without one or more components arranged therebetween to provide electrical continuity.

As illustrated in FIG. 6, when fabrication of the extended EAM die 500 is complete, a passivation layer may be added to the active area 502 of the extended EAM die 500 and a flex 602—partially represented in FIG. 6—may be coupled to the front side 501 of the extended EAM die 500 (and of the CMUT die), across the I/O region 506. The flex 602 may be a flex circuit that provides electrical connectivity away from the extended EAM die 500, to other electrical devices and components. The flex 602 may thus be directly coupled to the bias contacts 510 and the output contacts 508 to transmit electrical signals to and from the I/O region.

An example of an EAM 700 is shown in FIG. 7, the EAM 700 composed of four replicates 706, 708, 710, and 712 of the extended EAM die 500 of FIG. 6. The four replicates may have two different designs. In a first design, bias contacts may be included in replicates 708 and 710. In a second design, bias contacts may be included in replicates 706 and 712. The flexes 602 may be coupled to the EAM 700 along the I/O region of each extended EAM die 500, on opposite sides of the EAM 700. In one example, an overall width 702 of the EAM 700, along the x-axis, may be 25.8 mm and an overall height 704 of the EAM 700, along the y-axis, may be 16.3 mm.

As indicated in FIG. 6, each extended EAM die 500 may have a width 604 of 12.9 mm and a height 606 of 8.13 mm, in one example. A sub-width 608 of the I/O region 506 may be at least 1 mm and a clearance distance 610 may be provided between an inner edge of the flex 602 and an outer perimeter of the CMUTs 504. A presence of the clearance distance 610, which may be between 0.2-0.9 mm and in the depicted example is 0.64 mm, may be enabled due to extension of the areas of the ASIC dies, the clearance distance 610 mitigating bleeding of an organic material of the flex 602 in the CMUTs 504 during attachment of the flex 602 to the extended EAM tile 500.

Simplified cross-sectional views of the extended EAM are shown in FIGS. 8 and 9. A first cross-section obtained along line A-A′ in FIG. 5 is depicted in FIG. 8 and a second cross-section obtained along line B-B′ in FIG. 5 is depicted in FIG. 9. The cross-sectional views are simplified in that a representative, abbreviated number of the CMUT cells 504 and the output contacts 508 are shown and some layers of the extended EAM tile 500, such as metal layers and passivation layers, are omitted for brevity. The extended EAM die 500 may be formed of a CMUT die 802 arranged above, relative to the z-axis, and coupled to an ASIC tile 804. The CMUT die 802 and the ASIC tile 804 are shown spaced apart in FIGS. 8 and 9 for clarity but are in contact with one another when fabrication of the extended EAM die 500 is complete.

Furthermore, while FIGS. 8-9 are drawn approximately to scale along the x- and y-axes, the first and second cross-sections of FIGS. 8-9 are not drawn to scale along the z-axis. By fabricating the EAM die by a semiconductor approach, described further below, a thickness of the EAM may be reduced relative to a conventional EAM configuration where a CMUT wafer and an ASIC wafer are processed and diced separately. For example, the CMUT wafer of the conventional EAM configuration may be 300-400 μm thick (as defined along the z-axis) and the ASIC wafer may have a similar thickness. In the embodiment of the extended EAM tile shown and described herein, the CMUT wafer may be thinner, having a thickness of, for example, 30 μm, while a CMOS wafer from which the ASIC tiles are formed may be 300-400 μm thick. The CMUT wafer 802 of FIGS. 8 and 9 is therefore shown thicker than to scale in order to depict its features with greater clarity.

As shown in FIG. 8, the first cross-section includes the active area 502 separated from the I/O region 506 by the clearance distance 610. A perimeter of the extended EAM 500 along the x-axis, as defined by dicing along the z-y plane, is indicated by dashed lines 806. An extended area 808 of the extended EAM die 500, relative to a width of a conventional EAM tile is also indicated.

In the active area 502, the CMUT cells 504 may be located at an upper surface (relative to the z-axis) of the CMUT die 802, which is arranged at a front side of the EAM 700. A front side metal 810 may be deposited over each of the CMUTs 504 which may be electrically coupled to a bottom side contact 812 of each of the CMUTs 504 by conductive vias 814. The conductive vias 814 may be formed of an insulating layer 814a surrounding a conductive core 814b and may extend through a silicon base of the CMUT die 802. Oxide layers 816 of the CMUT die 802 may be etched to provide contact between the conductive core 814b of the conductive vias 814 and the CMUTs 504 and between the conductive core 814b of the conductive vias 814 and the bottom side contact 812 of the CMUTs 504. A patterned metallization layer 818 may be deposited at a bottom surface (relative to the z-axis) of the CMUT die 802 between the conductive core 814b and the bottom side contacts 812a, which may enable transfer of electrical energy between the conductive core 814b and the bottom side contacts 812a. The conductive vias 814b may therefore extend through at least a portion of a thickness (as defined along the z-axis) of the CMUT die 802 to connect the front side metal 810 to the bottom side contact 812 for each of the CMUT cells 504.

The bottom side contact 812 may protrude from the bottom surface of the CMUT die 802 and may be offset along the z-axis from the CMUT 504 that the respective bottom side contact 812 is electrically coupled to. The bottom surface of the CMUT die 802 may also include bias planes 821 arranged between the bottom side contacts 812. The bias planes 821 may be electrically coupled to a bias contact (e.g., bias contact 910 of FIG. 9 or bias contacts 510 of FIGS. 5 and 6) located at the upper surface of the CMUT die 802. The bias planes 821 may optionally be coupled to the bias contacts by vias.

The bottom side contact 812 of each of the CMUT cells 504 may be arranged in face-sharing contact with a respective ASIC pad 820 of the ASIC tile 804. The ASIC pad 820 may include a metal contact 820a and a metal layer 820b for wafer-to-wafer bonding. In one embodiment, metal contact 820a may be constructed of gold, with eventually a metallic barrier layer like in between 820a and 820b, and metal layer 820b may be constructed of aluminum. The bottom side contact 812 of each of the CMUTs 504 is therefore in direct contact with the respective ASIC pad 820 of the ASIC tile 804 without any other components or layers arranged in between. As a result, a distance between the CMUTs 504 and the ASIC tile 804 is minimized which decreases losses incurred during signal transmission between the CMUT cells 504 and the ASIC contact 820, and reduces a number of components, and therefore a cost, of the extended EAM die 500.

The upper surface of the CMUT die 802 does not include any CMUT cell 504 along the clearance distance 610 and the I/O region 506. In the I/O region 506, the output contacts 508 each include an upper RDL 508a and a lower RDL 508b, with the upper and lower RDLs 508a, 508b electrically coupled by the conductive vias 814. One or more interfacing contacts 822 arranged at the bottom surface of the CMUT die 802 in the I/O region 506 may be electrically coupled to each of the output contacts 508 through the upper and lower RDLs 508a, 508b, and the conductive vias 814b.

A column (e.g., as defined along the y-axis) of the output contacts 508 adjacent and proximate to the clearance distance 610 may have lower RDLs 508b that are elongated to extend along both the x- and y-axes towards the active area 502. Each of the output contacts 508 of the column proximate to the clearance distance 610 may be electrically coupled to both the interfacing contact 822 directly below the respective output contact 508 and to an additional interfacing contact 822 located in the clearance distance 610.

Each of the interfacing contacts 822 may be in face-sharing contact with the ASIC pads 820 aligned therewith along the z-axis. By extending the EAM tile 500 along the x-axis by the extended area 808, the output contacts 508 may be positioned in the I/O region 506 at the upper surface of the CMUT die 802, allowing the output contacts 508 to be accessed at the front side of the EAM 700, rather than routed outwards from the EAM 700 along its outer perimeter by, for example, wire bonding. As described above, the EAM die 500 may also be extended along the y-axis, as indicated in the second cross-section of FIG. 9.

The CMOS wafer may be diced along the y-x plane as indicated by dashed lines 902 to form the extended EAM die 500, which may be extended relative to a conventional ASIC die by an extended area 904. The extended EAM die 500 may optionally be further diced as indicated by dashed line 906, which removes a dummy contact 908 from the EAM tile 500 and decreases a footprint of the EAM 500 along the y-axis. It will be appreciated that the EAM die 500 may also include some dummy contacts 908 along the x-axis located proximate to an edge of the EAM tile 500, as shown in FIG. 9. The reduced footprint of the EAM 500 may be desirable for, as one example, intercostal applications.

In other examples, the CMUT die 802 may include more or less than one dummy contact 908 along the y-axis, depending on a desired number of columns of output contacts (e.g., the output contacts 508 of FIGS. 5-6.) As such, by configuring the CMUT wafer with dummy contacts, a number of columns of output contacts (and of dummy contacts) of the CMUT die may differ from a number of columns of ASIC pads in the I/O region. The CMUT wafer may thereby be readily adapted to couple to the ASIC tile regardless of a desired number of output contact columns on the EAM tile.

The bottom side contacts 812 of the CMUT die 802 may be in face-sharing contact with the ASIC pads 820 as described above. The CMUT die 802 may further include a bias contact 910. The bias contact 910 may enable polarization of a plane 821 of the CMUT cells 504 through the front side of the EAM die 500.

An EAM formed of extended EAM dies, such as the extended EAM tile 500 of FIGS. 5-9, may be fabricated by a method 1000 depicted in FIG. 10. The method 1000 may be executed by a technician, by an automated system, or by a combination of both, in conjunction with various manufacturing equipment and devices. In method 1000, the EAM may be manufactured as a MEMS device. Processes described in method 1000 are illustrated in FIGS. 11-14, which are described in conjunction with discussion of method 1000. It will be appreciated that method 1000 is a non-limiting example of how the EAM may be fabricated. Other techniques and/or variation in the processes and/or in an order of the processes elaborated in method 1000 are possible without departing from the scope of the present disclosure.

Method 1000 includes partial fabrication of a CMUT wafer (1002-1014) and preparation of an ASIC die (1016-1022), which may or may not occur concurrently. For example, the fabrication of the CMUT wafer and the preparation of the ASIC wafer may be processes that are executed independent of one another. The processes may therefore be performed according to any timing relative to one another. As an example, the CMUT wafer may be fabricated before the ASIC wafer is prepared or the ASIC wafer may be prepared before the CMUT wafer is fabricated. As yet another example, the CMUT wafer may be fabricated while the ASIC wafer is prepared.

At 1002, method 1000 includes fabricating partially the CMUT wafer, which may be a MEMS wafer. The CMUT wafer may be partially fabricated because the CMUT wafer may be thinned down and the top contacts made in a second, later part of the fabrication of the CMUT wafer. The CMUT die may have dimensions corresponding to a designated, (e.g., already existing, ASIC die diced from a CMOS wafer) or the dimensions of the CMUT die may correspond to a tiled configuration of the ASIC die. An EAM tile formed of the CMUT die and the ASIC die may have an extended footprint relative to conventional EAM tiles along at least one dimension across a plane of the EAM tile, where ASIC die of the conventional EAM tile may be double diced with dicing lanes which are subsequently removed. The CMUT wafer may be fabricated with a pitch that corresponds to a pitch of ASIC pads of the ASIC die.

At 1004, fabricating the CMUT wafer includes oxidizing a first wafer, which may be a SOI (Silicon on Insulator) wafer. The first wafer may have a controlled, target thickness and may be used as a handle during fabrication of the EAM, until the thickness of the first wafer is reduced, as described further below. An example of a SOI wafer 1100 that may be used in method 1000 to fabricate the CMUT wafer is illustrated in FIG. 11.

Referring to FIG. 11, an SOI wafer 1100 may have a thickness 1106, including an embedded silicon oxide layer 1104 having a controlled, target thickness and a highly doped silicon layer 1108 having a controlled thickness below oxide layer 1104, with respect to the Z axis. The SOI wafer 1100 may have outer silicon oxide layers 1102 extending along upper and lower surfaces (with respect to the z-axis) which may be formed during oxidation of the SOI wafer 1100 at 1004 of method 1000.

Returning to FIG. 10, at 1006 of method 1000, a second, highly doped silicon wafer may be processed by forming conductive vias that extend into a thickness of the second, highly doped silicon wafer. Processing of the second silicon wafer at 1006 to form the conductive vias may occur concurrent with, before, or after oxidation of the first wafer. As an example, the conductive vias may be formed using deep reactive ion etching (DRIE) to create trenches that extend vertically (along the z-axis) into the highly doped silicon wafer. The trenches may be blind trenches that extend into a portion of the thickness of the second silicon wafer, as shown in FIG. 11, or may be through trenches that extend through the entire thickness of the second silicon wafer.

Forming the conductive vias may include oxidation and via fill with an insulator in the trenches formed in the second silicon wafer by DRIE. The layers may be deposited in the trench by, as one example, physical vapor deposition (PVD). Growth of the seed layer to fill the trenches may be subsequently promoted, e.g., after oxidation of the second silicon wafer at 1008. However, in other examples, the conductive via trenches may be formed at a later point during the fabrication of the CMUT wafer or as an end step in the fabrication of the CMUT wafer.

At 1008, method 1000 includes oxidizing the second, highly doped silicon wafer and filling the trenches of the conductive vias with an insulating material to form the conductive vias. Furthermore, at 1008, an additional oxide layer may be deposited by physical vapor deposition (PECVD) and cavities may be formed at a surface of the oxidized second silicon wafer, such as at an upper surface of the second silicon wafer that interfaces with the first wafer. The cavities may be formed according to known techniques for forming cavities for CMUT cells. An example of a highly doped silicon wafer 1110, shown after creation of the conductive vias, oxidation, and cavity formation, is illustrated in FIG. 11.

The highly doped silicon wafer 1110 of FIG. 11 may have a standard thickness 1112 of, for example, 400 μm. An upper surface of the highly doped silicon wafer 1110 (with respect to the z-axis) may include cavities 1114, formed as described above at 1006 of method 1000. The highly doped silicon wafer 1110 may have outer silicon oxide layers 1116 extending across the upper surface and a lower surface of the highly doped silicon wafer 1110 and the cavities 1114 may be disposed in the outer silicon oxide layer 1116 at the upper surface. In addition, the highly doped silicon wafer 1110 may have conductive vias 1118 extending from the upper surface of the highly doped silicon wafer 1110 into a portion of the thickness 1112 of the highly doped silicon wafer 1110.

Returning to FIG. 10, at 1010, method 1000 includes bonding the first, SOI wafer and the second, highly doped silicon wafer to one another. For example, the first wafer and the second silicon wafer may undergo oxide-oxide fusion bonding and fusion bonding annealing to adhere the wafers to one another. The fusion bonding annealing may be conducted at temperatures greater than 1000° C., in one example.

At 1012, the thickness of the second silicon wafer may be reduced by grinding the bottom of the second silicon wafer. An amount of the second, highly doped silicon wafer that is removed may be an amount that exposes ends of the conductive vias. For example, 370 μm may be removed from the thickness of the second silicon wafer. The bottom surface of the second silicon wafer, after grinding, may be treated at 1014 by depositing layers thereat. The layers may include a first passivation layer in contact with the ends of the conductive vias, where the passivation layer may be patterned after deposition. The layers may further include a metal layer which is also patterned after deposition, and an optional second passivation layer. Bottom side contacts may then be formed at the bottom surface of the second silicon wafer. For example, the bottom side contacts may include a first layer of aluminum and second, thicker layer of gold. A barrier layer, such as nickel, titanium tungstate, or titanium nitride, may be included between the first and second layers to inhibit interdiffusion of the metals during wafer-to-wafer bonding. In addition, dummy contacts may be formed to provide mechanical support for the extended EAM die along extended, peripheral areas of the EAM tile. Further, bias components, such as bias contacts, and bias planes may be formed at 1014.

An example of a CMUT wafer 1200 fabricated according to 1002-1014 of method 1000 is illustrated in FIG. 12. The CMUT wafer 1200 is composed of the SOI wafer 1100 bonded to the highly doped silicon wafer 1110 such that the bottom surface of the SOI wafer 1100 is in face-sharing contact with and bonded to the upper surface of the highly doped silicon wafer 1110. The cavities 1114 may be enclosed within the layers of silicon oxide 1102 and 1116 of the wafers.

The thickness 1106 of the SOI wafer 1100 is unchanged relative to FIG. 11 but the thickness 1112 of the highly doped silicon wafer 1110 is reduced such that the conductive vias 1118 extend through the entire thickness 1112 of the highly doped silicon wafer 1110. At the bottom surface of the highly doped silicon wafer 1110, the CMUT wafer 1200 may include at least one patterned passivation layer 1202 formed of an inert material, such as a metal oxide or nitride. The bottom surface may also include a metal layer 1204 for providing electrical continuity from the upper surface to the bottom surface of the highly doped silicon wafer 1110 through the conductive vias 1118, and a bias plane 1206 for delivering a bias voltage to CMUT cells to be formed in the CMUT wafer 1200. The metal layer 1204 and the bias plane 1206 may each be in direct contact with one of the conductive vias 1118. Bottom side contacts 1208, similar to the bottom side contacts 812 of FIGS. 8-9, may be formed under (relative to the z-axis) the passivation layer 1202.

Returning to FIG. 10, an ASIC wafer may be prepared for coupling to the CMUT wafer to form the EAM wafer at 1016 of method 1000. At 1018, preparing the ASIC wafer includes depositing at least one passivation layer and flattening a surface of the ASIC wafer. The passivation layer may be deposited by plasma enhanced chemical vapor deposition (PECVD) and the deposited passivation layer may be flattened using chemical-mechanical polishing (CMP), as one example. The passivation layer is etched at 1020 of method 1000 to provide access to ASIC pads of the ASIC dies which may be covered by the passivation layer during deposition of the passivation layer.

At 1022, preparing the ASIC wafer further includes depositing metal onto a passivated and flattened upper surface of the ASIC wafer. The metal may be selected according to a desired interaction of the metal with the bottom side contacts of the CMUT wafer for bonding therebetween. For example, an adhesion layer of metal may first be applied followed by a seed layer of metal. In one example, the adhesion layer of metal may be titanium, a titanium tungsten alloy, titanium nitride, or a combination thereof, and the seed layer of metal may be gold, which may be deposited by sputtering. The seed layer of metal may form an ASIC contact, e.g., the metal contact 820b of FIGS. 8-9. Dummy contacts (e.g., dummy contact 1312 depicted in FIG. 13) may also be formed at the upper surface of the ASIC die to mechanically support the CMUT die in an I/O region, such as the I/O region 506 of FIGS. 5-6, as shown in FIG. 13.

An ASIC wafer 1300 prepared according to 1016-1022 of method 1000, for bonding to the CMUT wafer 1200, is illustrated in FIG. 13. An upper surface (relative to the z-axis) of the ASIC wafer 1300 may include a first passivation layer 1302 and a second passivation layer 1304 stacked over the first passivation layer 1302, where the second passivation layer 1304 may be an optional layer. An ASIC pad 1306 of the ASIC die 1300 may include an adhesion layer of metal 1308 and a metal contact 1310, formed of a seed metal. The dummy contact 1312 may also be included at the upper surface of the ASIC die 1300, along a periphery of the ASIC die 1300.

Returning to FIG. 10, method 1000 includes bonding the CMUT wafer and the ASIC wafer to one another at 1024 to form the EAM wafer. As an example, gold-gold thermocompression bonding may be used to adhere the bottom side contacts of the CMUT wafer to the metal contacts of the ASIC wafer. For example, as shown in FIG. 13, the bottom side contacts 1208 of the CMUT wafer 1200 may be placed in face-sharing contact with the metal contacts 1310 of the ASIC wafer 1300 and bonded to one another.

At 1026 of method 1000, the thickness of the first SOI wafer is thinned by, for example, grinding down to the second, highly doped silicon layer (e.g., the highly doped silicon layer 1108 of FIGS. 11-13), following by etching of the oxide layer to form membranes over the cavities (e.g., CMUT membranes). A front side of the EAM is metallized by depositing a metal over the membranes, such as aluminum or an aluminum alloy. The metal may be patterned to provide electrical contacts between the membranes and the conductive vias. A passivation layer is deposited over the metal and patterned at 1028. Further, at 1028, output and bias contacts, such as the output contacts 508 and the bias contacts 510 of FIG. 5, are formed at the front side of the EAM. At 1030 of method 1000, the EAM is arranged relative to other EAM tiles according to a desired array for an EAM of a probe.

A final configuration of an extended EAM 1400 is illustrated in FIG. 14, where the EAM is formed by bonding the CMUT wafer 1200 of FIGS. 12-13 to the ASIC wafer 1300 of FIG. 13. The thickness 1106 of the first SOI wafer 1100 is reduced relative to FIGS. 11-13 such that the embedded silicon oxide layer 1104 is removed. Membranes 1402 may be formed over the cavities 1114 from the highly doped silicon layer 1108 (as shown in FIGS. 11-3) at a front side 1404 of the extended EAM tile 1400. A metal layer 1406 may be deposited over the membranes 1402 and may be in contact with metal contacts 1410 of conductive vias 1412 of the extended EAM 1400. A bias contact 1408 may be formed over the dummy contact 1312 of the ASIC die 1300, with respect to the z-axis. Formation of output and bias contacts are thereby defined based on final passivation of the EAM and a positioning thereof along a periphery of the EAM allows the output and bias contacts to be compatible with attachment of flex circuits.

In this way, a development time and cost for fabricating EAMs may be decreased using a reproducible and scalable process. The process includes preparing a MEMS wafer, e.g., a CMUT wafer, and a CMOS wafer, e.g., an ASIC wafer, for bonding to one another to form an extended EAM tile. The extended EAM tile may be arranged in an array of an EAM for a probe and may have an extended area compared to an EAM tile configured with an ASIC common to the extended EAM tile but diced in a conventional manner. By extending the area of the extended EAM tile, an I/O region may be disposed along a periphery of a front side of the EAM tile, adjacent to its active area, with output contacts and bias contacts positioned in the active area. The output and bias contacts may be redistributed from an upper surface of the ASIC die, where access to the output and bias contacts may otherwise be constrained. The extended EAM tile may be fabricated at a wafer scale, enabling a cost-effective high volume manufacturing approach to be utilized that relies on well-known wafer-to-wafer bonding techniques and without imposing additional manufacturing steps used for manufacturing MEMS devices. Further, a number of output contacts at the front side of the EAM tile may be independent of a number of ASIC pads within the I/O region.

FIGS. 2-9 and 11-14 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example. FIGS. 3-7 are shown approximately to scale.

As used herein, an element or step recited in the singular and proceeded with the word “a” or “an” should be understood as not excluding plural of said elements or steps, unless such exclusion is explicitly stated. Furthermore, references to “one embodiment” of the present disclosure are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The disclosure also provides support for an ultrasound probe, comprising an electroacoustic module formed of a microelectromechanical systems (MEMS) wafer bonded to a complementary metal-oxide semiconductor (CMOS) wafer as an Application-Specific Integrated Circuit (ASIC) and having a front side formed of a surface of the MEMS wafer, the surface of the MEMS wafer including an active area of capacitive micromachined ultrasound transducer (CMUT) cells and input/output (I/O) regions of input and output contacts of ASIC which are redistributed to the surface of the MEMS wafer arranged adjacent to the active area. In a first example of the system, the CMOS wafer is diced along no more than one direction to form ASIC dies, and wherein dimensions of the MEMS wafer along a plane of the MEMS wafer is equal to dimensions of the CMOS wafer, along a plane of the CMOS wafer. In a second example of the system, optionally including the first example, the I/O regions are located along a periphery of the electroacoustic module, on opposite sides of the active area, and wherein the I/O regions further includes CMUT bias contacts. In a third example of the system, optionally including one or both of the first and second examples, the I/O regions are configured to be coupled to interconnecting circuits extending away from the active area. In a fourth example of the system, optionally including one or more or each of the first through third examples, clearance distances are provided between the active area and the I/O regions, and wherein no CMUT cells or output contacts are positioned in the clearance distances. In a fifth example of the system, optionally including one or more or each of the first through fourth examples, the I/O regions further include dummy contacts at an underside of the MEMS wafer and an upper surface of the CMOS wafer, the dummy contacts configured to provide mechanical support to the electroacoustic module along a periphery of the electroacoustic module. In a sixth example of the system, optionally including one or more or each of the first through fifth examples, the MEMS wafer is directly coupled to the CMOS wafer without an interposer or flex circuits arranged therebetween. In a seventh example of the system, optionally including one or more or each of the first through sixth examples, a thickness of the MEMS wafer is less than a thickness of the CMOS wafer.

The disclosure also provides support for a method, comprising, fabricating a capacitive micromachined ultrasound transducer (CMUT) wafer with bottom side contacts configured to mate with pads of an ASIC die, coupling the CMUT wafer to the ASIC die to form an electroacoustic module (EAM) stack having output contacts at a front side of the CMUT wafer, and dicing the EAM stack along no more than one axis across a plane of the EAM stack to form an EAM tile. In a first example of the method, fabricating the CMUT wafer includes bonding a silicon-on-insulator (SOI) wafer to a highly doped silicon wafer, the SOI wafer having an embedded silicon oxide layer and a highly doped silicon layer and the highly doped silicon wafer having cavities etched into a silicon oxide layer of the highly doped silicon wafer. In a second example of the method, optionally including the first example, fabricating the CMUT wafer includes forming trenches through at least a portion of a thickness of the highly doped silicon wafer and filling the trenches to form conductive vias. In a third example of the method, optionally including one or both of the first and second examples, the thickness of the highly doped silicon wafer is reduced, after bonding the SOI wafer and the highly doped silicon wafer to one another, to expose ends of the conductive vias at a bottom surface of the highly doped silicon wafer, and a plurality of layers, the plurality of layers including at least one passivation layer, a bias plane, and a metal layer, are deposited and patterned at the bottom surface. In a fourth example of the method, optionally including one or more or each of the first through third examples, metal contacts are formed at the bottom surface of the highly doped silicon wafer after the plurality of layers are deposited and patterned. In a fifth example of the method, optionally including one or more or each of the first through fourth examples, the method further comprises: preparing the ASIC wafer prior to coupling to the CMUT wafer by depositing a passivation layer over an upper surface of the ASIC die, the upper surface including ASIC pads, and flattening the upper surface. In a sixth example of the method, optionally including one or more or each of the first through fifth examples, the method further comprises: etching the passivation layer to provide access to the ASIC pads prior to coupling the CMUT wafer to the ASIC wafer. In a seventh example of the method, optionally including one or more or each of the first through sixth examples, the CMUT wafer is bonded to the ASIC wafer by gold-to-gold thermocompression bonding. In a eighth example of the method, optionally including one or more or each of the first through seventh examples, the method further comprises: reducing a thickness of the EAM to form CMUT membranes at the front side of the EAM, forming the output contacts and bias contacts over the front side, and optionally, depositing a passivation layer over the front side.

The disclosure also provides support for an electroacoustic module (EAM) comprising a plurality of tiles, each tile comprising: an array of capacitive micromachined ultrasound transducers (CMUTs), an application-specific integrated circuit (ASIC) arranged below the array of CMUTs and electrically coupled to the array of CMUTs, and output contacts positioned adjacent to the array of CMUTs, along a periphery of the respective tile and at a front side of the tile. In a first example of the system, the output contacts are arranged in an input/output (I/O) region adjacent to the array of CMUTs, and wherein a number of columns of the output contacts is not equal to a number of columns of ASIC pads of the ASIC in the I/O region. In a second example of the system, optionally including the first example, the ASIC is compatible for electrical coupling with more than one configuration of the array of CMUTs.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An ultrasound probe, comprising;
an electroacoustic module formed of a microelectromechanical systems (MEMS) wafer bonded to a complementary metal-oxide semiconductor (CMOS) wafer, as an Application-Specific Integrated Circuit (ASIC), and having a front side formed of a surface of the MEMS wafer, the surface of the MEMS wafer including an active area of capacitive micromachined ultrasound transducer (CMUT) cells and input/output (I/O) regions of input and output contacts of ASIC which are redistributed to the surface of the MEMS wafer arranged adjacent to the active area.

2. The probe of claim 1, wherein the CMOS wafer is diced along no more than one direction to form ASIC dies, and wherein dimensions of the MEMS wafer along a plane of the MEMS wafer is equal to dimensions of the CMOS wafer, along a plane of the CMOS wafer.

3. The probe of claim 1, wherein the I/O regions are located along a periphery of the electroacoustic module, on opposite sides of the active area, and wherein the I/O regions further includes CMUT bias contacts.

4. The probe of claim 1, wherein the I/O regions are configured to be coupled to interconnecting circuits extending away from the active area.

5. The probe of claim 4, wherein clearance distances are provided between the active area and the I/O regions, and wherein no CMUT cells or output contacts are positioned in the clearance distances.

6. The probe of claim 1, wherein the I/O regions further include dummy contacts at an underside of the MEMS wafer and an upper surface of the CMOS wafer, the dummy contacts configured to provide mechanical support to the electroacoustic module along a periphery of the electroacoustic module.

7. The probe of claim 1, wherein the MEMS wafer is directly coupled to the CMOS wafer without an interposer or flex circuits arranged therebetween.

8. The probe of claim 1, wherein a thickness of the MEMS wafer is less than a thickness of the CMOS wafer.

9. A method, comprising;
fabricating a capacitive micromachined ultrasound transducer (CMUT) wafer with bottom side contacts configured to mate with pads of an ASIC wafer;
coupling the CMUT wafer to the ASIC wafer to form an electroacoustic module (EAM) stack wafer having output contacts at a front side of the CMUT wafer; and
dicing the EAM stack wafer along no more than one axis across a plane of the EAM stack to form an EAM tile.

10. The method of claim 9, wherein fabricating the CMUT wafer includes bonding a silicon-on-insulator (SOI) wafer to a highly doped silicon wafer, the SOI wafer having an embedded silicon oxide layer and a highly doped silicon layer and the highly doped silicon wafer having cavities etched into a silicon oxide layer of the highly doped silicon wafer.

11. The method of claim 10, wherein fabricating the CMUT wafer includes forming trenches through at least a portion of a thickness of the highly doped silicon wafer and filling the trenches with an insulator to form conductive vias.

12. The method of claim 11, wherein the thickness of the highly doped silicon wafer is reduced, after bonding the SOI wafer and the highly doped silicon wafer to one another, to expose ends of the conductive vias at a bottom surface of the highly doped silicon wafer, and a plurality of layers, the plurality of layers including at least one passivation layer, a bias plane, and a metal layer, are deposited and patterned at the bottom surface.

13. The method of claim 12, wherein metal contacts are formed at the bottom surface of the highly doped silicon wafer after the plurality of layers are deposited and patterned.

14. The method of claim 9, further comprising preparing the ASIC wafer prior to coupling to the CMUT wafer by depositing a passivation layer over an upper surface of the ASIC wafer, the upper surface including ASIC pads, and flattening the upper surface.

15. The method of claim 14, further comprising etching the passivation layer to provide access to the ASIC pads prior to coupling the CMUT wafer to the ASIC wafer.

16. The method of claim 10, wherein the CMUT wafer is bonded to the ASIC wafer by gold-to-gold thermocompression bonding.

17. The method of claim 10, further comprising reducing a thickness of the EAM to form CMUT membranes at the front side of the EAM, forming the output contacts and bias contacts over the front side and optionally, depositing a passivation layer over the front side.

18. An electroacoustic module (EAM) comprising a plurality of tiles, each tile comprising:
an array of capacitive micromachined ultrasound transducers (CMUTs);
an application-specific integrated circuit (ASIC) arranged below the array of CMUTs and electrically coupled to the array of CMUTs; and
output contacts positioned adjacent to the array of CMUTs, along a periphery of the respective tile and at a front side of the tile.

19. The EAM of claim 18, wherein the output contacts are arranged in an input/output (I/O) region adjacent to the array of CMUTs, and wherein a number of columns of the output contacts is not equal to a number of columns of ASIC pads of the ASIC in the I/O region.

20. The EAM of claim 18, wherein the ASIC is compatible for electrical coupling with more than one configuration of the array of CMUTs.
